(19) United States
(12) Patent Application Publication
(10) Pub. No.: US 2022/0370031 A1
(43) Pub. Date: Nov. 24, 2022

(54) AUTOMATED SYSTEM AND METHOD OF MONITORING ANATOMICAL STRUCTURES

(71) Applicants: NGEE ANN POLYTECHNIC, Singapore (SG); SINGAPORE HEALTH SERVICES PTE LTD, Singapore (SG); KUMAMOTO UNIVERSITY, Kumamoto-shi (JP)

(72) Inventors: Rajendra Udyavara ACHARYA, Singapore (SG); Ru San TAN, Singapore (SG); Makiko KOBAYASHI, Kumamoto-shi (JP); Masayuki TANABE, Kumamoto-shi (JP); Toshitaka YAMAKAWA, Kumamoto-shi (JP)

(21) Appl. No.: 17/761,756

(22) PCT Filed: Sep. 21, 2020
(86) PCT No.: PCT/SG2020/050538

§ 371 Date: Mar. 18, 2022

Related U.S. Application Data
(60) Provisional application No. 62/902,926, filed on Sep. 19, 2019.

(51) Int. Cl. A61B 8/06; G06T 7/00; G16H 50/30

(52) U.S. Cl. CPC A61B 8/06; G06T 7/0012; G16H 50/30; G06T 2207/10132

(57) ABSTRACT

Embodiments include a patch-type, ultrasound sensor system and method to monitor the function and motion of a patients anatomical structure, comprising processing at least one received ultrasound image using one or more analytical tools, including radon transformation, higher-order spectra techniques, and/or active contour models, to generate at least one processed ultrasound image; inputting the at least one processed ultrasound image into a deep learning Convolutional Neural Network to obtain an automatic classification result selected from two or more classes indicating the functional state of the anatomical structure. The patch-type, ultrasound sensor system can communicate via a wireless or wired connection. The monitoring can be at rest or during surgery or other procedure or whilst the subject is exposed to any physiological stressors as part of medical examinations, and can be adapted for use in monitoring the function of body structures including the heart, blood vessels, lungs or joints.

### CROSS REFERENCE TO RELATED APPLICATIONS

The present application claims priority to U.S. Provisional Application No. 62/902,926 filed 19 Sep. 2019, the contents of which are incorporated by reference.

### TECHNICAL FIELD

The present disclosure relates generally to diagnostic and therapeutic medical imaging, and more specifically to a computer implemented system and method to monitor the function of anatomical structures of a subject.

### BACKGROUND

Ultrasonic images, also known as sonograms, are made by sending pulses of ultrasound into tissue using a probe. The ultrasound pulses echo off tissues with different reflection properties and are recorded and displayed as an image. Medical ultrasound (also referred to as diagnostic sonography or ultrasonography) refers to diagnostic imaging or therapeutic application of ultrasound. Ultrasound can create an image of internal body structures such as tendons, muscles, joints, blood vessels, and internal organs. Its aim is often to find a source of a disease or to exclude pathology. Obstetric ultrasound was an early development and application of clinical ultrasound which is common today.

Ultrasound has several advantages over other diagnostic methods. It is non-invasive and provides images in real time. Moreover, modern machines are portable and can be brought to the bedside. It is substantially lower in cost than other imaging modalities and does not use harmful ionizing radiation.

Numerous ultrasound sensor devices have been developed to assess the structure and function of internal organs, muscle or tissue within the human body to assist in identifying conditions or diseases or the likelihood of the development of such conditions or diseases. These ultrasound devices typically utilize data from multiple short scans lasting seconds acquired during a single examination session lasting several minutes. For the same purpose mentioned above, the assessment can also be performed over a short duration (typically minutes) before, during and after the administration of a stressor. For example, ultrasound can be conducted on a human subject during exercise (e.g. exercise stress echocardiography), ischemia-reperfusion (e.g. by compressive occlusion of blood flow of the brachial artery during flow-mediated dilatation testing), heat/cold application, as well as during surgery (e.g. intraoperative echocardiography), or other procedures. However, it has not been feasible to perform ultrasound on the human body for extended periods beyond an hour or remotely because of the need to maintain constant pressure contact of the sensor on the human body part of interest.

Traditional ultrasound sensor devices can provide live images and enable extraction of characteristic features using signal processing techniques. For example, in cardiology, ultrasound is a ubiquitous and versatile technique that allows real-time imaging of the heart and blood vessels for assessment of cardiovascular health. The ultrasound probe is placed on the skin overlying the heart or blood vessel of interest during the test. The signal obtained by the probe is transmitted via a wire that attaches the ultrasound probe to the scanner, which processes the signal to produce images. While the device is portable, diagnostic information can only be garnered at the time of the scan. Conventional devices are limited to producing single still images or videos of moving structures of short duration at instances that the ultrasound scanner is activated. Thus, the ability to non-invasively monitor the motion of an organ, such as the heart, continuously over an extended period would have obvious advantages. An improved device should also allow remote continuous scanning via wireless connection such that the probe or ultrasound source can transmit data to a computer at a different location without the need to recharge over the extended duration of scanning.

US patent application US20120065479A1 discloses a wearable patch for use on the body which comprises an ultrasound sensor (preferably sensor array), a transmission system coupled to the ultrasound sensor adapted to provide signal information for ultrasound transmission into the body, and a receiver system coupled to the ultrasound sensor adapted to receive signal information from the reflected ultrasound signal received from the body. A control circuitry is coupled to the transmission system and the receiver system. The patch is preferably provided with a wireless communication system to permit external control and/or communication. The patch enables continuous monitoring of the heartbeat without interfering with the patient's routine activities. Applications include but are not limited to diagnostics and monitoring, rehabilitation and wound healing. While this is an improvement over conventional ultrasound techniques, it has limitations if deployed without the ability analyze the large amounts of data that the patch may continuously collect and store. The data must be analyzed by trained specialists which is both time consuming and prone to subjectivity.

The signals and data obtained from conventional ultrasound sensor devices can be processed through several ways for extracting measurements and classifying results depending on the medical application. Conventional methods of processing ultrasound sensor signal data may be inconsistent due to the inherent requirement for some level of human manual input, for instance, the level of noise reduction threshold. In this regard, deep learning techniques have been used extensively in various studies to process and classify ultrasound sensor signal data. Deep learning Convolutional Neural Network (CNN) are currently used in the medical field for processing and analyzing signals from medical sensors to increase the processing speed and to provide results that assist in identifying conditions or diseases in an efficient manner.

A need, therefore, exists for an improved automatic, computed implemented system and method to non-invasively assess anatomical structures within a human body, and with the option to continuously monitor signals over extended durations, in order to process and accurately classify received ultrasound signals, including using the deep learning CNN.

### SUMMARY

The following summary is provided to facilitate an understanding of some of the innovative features unique to the disclosed embodiment and is not intended to be a full description. A full appreciation of the various aspects of the embodiments disclosed herein can be gained by taking into consideration the entire specification, claims, drawings, and abstract as a whole.

Embodiments include a patch-type, ultrasound sensor system and method to monitor the function of a subject's anatomical structure to classify received ultrasound signals using the deep learning CNN. The monitoring can be while the subject is at rest, in response to stressor/s, or during surgery or other procedures. In particular, the system and method disclosed herein can be adapted for use in monitoring the function of the heart or blood vessels as well as other body structures, including but not limited to lungs, tissue and joints.

In one embodiment, there is provided a system for assessing and monitoring an anatomical structure of a subject, comprising: at least one ultrasound patch attached to said subject, wherein said patch comprises one or more ultrasound sensors, communication system, and an electric board for ultrasound transmission and/or reception, wherein the ultrasound patch generates at least one ultrasound image in one or more modes selected from the group consisting of M-mode, two-dimensional (2D), three-dimensional (3D) and Doppler ultrasound; a server comprising a cloud system for processing the at least one ultrasound image using one or more analytical tools to generate at least one processed ultrasound image, wherein the one or more analytical tools comprise radon transformation, higher-order spectra (HOS) techniques, and/or active contour model; a storage medium configured to store instructions defining a deep learning CNN, wherein the server executes the deep learning CNN to obtain an automatic classification result selected from two or more classes, to indicate the functional state of the anatomical structure; and an output to communicate the classification result to a user.

In one embodiment, the at least one processed ultrasound image is classified into two classes of either “normal” or “abnormal”.

In one embodiment, the at least one ultrasound patch generates the at least one ultrasound image in one or more modes selected from the group consisting of M-mode, 2D, 3D and Doppler ultrasound. It will be appreciated that while ultrasound echoes can be acquired continuously and images generated continually and stored throughout the time that the ultrasound sensor is being applied, the image output can also be stored in the form of a “still” image or “moving” images in video format (“cine”) at the discretion of the clinician depending on the medical need as well as storage and/or analytic capacities. The duration of the “moving” image is typically up to a few or several seconds that is deemed sufficient to depict the phasic motion of the structure of interest, e.g. the duration of one to 10 heart cycles is deemed sufficient for examining the beating heart in cardiac ultrasound in standard clinical applications. A single “still” ultrasound image can be a stored 2D image of the structure captured at one finite period in time. Alternatively, a “still” image can also capture one-dimensional spatial and/or Doppler-derived velocity information that is acquired over a time period, typically a few or several seconds, that is deemed sufficient to depict the phasic motion of the structure of interest. In particular, M-mode ultrasound depicts one-dimensional spatial information on the y-axis against time on the x-axis, while spectral Doppler ultrasound depicts velocity information on the y-axis against time on the x-axis.

Data to generate the at least one ultrasound image can also be acquired continuously over a predetermined time period, e.g. at least 15 seconds up to 24 hours, that is longer than the typical duration of a conventional “cine” scan to constitute “time-series data” that can itself be divided into segments of smaller time-series data sets of shorter durations. “Time-series data” can be stored and displayed in a video format and can comprise images depicting 2D spatial information with or without an overlay of Doppler-derived velocity information acquired over time, or images depicting 3D spatial information with or without an overlay of Doppler-derived velocity information acquired over time. Alternatively, “time-series data” can also comprise stored “still” images that display one-dimensional spatial and/or Doppler-derived velocity information on the y-axis against the acquisition time on the x-axis, such as M-mode ultrasound or spectral Doppler ultrasound. In this regard, “time-series data” are useful for characterizing and quantifying structural and functional changes in the structure of interest before, during and after the application of a stressor or the administration of a therapy.

Accordingly, in one embodiment the at least one ultrasound image can represent a time-series data set based upon structural (i.e., spatial) information over a time period. In one embodiment, the at least one ultrasound image can be an M-mode image that represents a time-series data set of the anatomical structure over a predetermined time period. The predetermined time period can be at least 15 seconds up to 24 hours. However, this may be modified dependent on the structure to monitor, the subject, and/or the circumstance of the assessment and monitoring.

In one embodiment, the at least one ultrasound patch comprises a thin and flexible piezoelectric material.

In one embodiment, the ultrasound patch is flexible and conforms to the surface of the subject's skin. However, it will be appreciated that in one embodiment the ultrasound patch can be modified and adapted to be attached to and conform with the surfaces of internal body cavities of a subject. In another embodiment, the ultrasound patch can be modified and adapted to operate as an implantable sensor.

In one embodiment, the ultrasound image is an M-mode, 2D echo, 3D echo or Doppler echo image.

In one embodiment, the one or more analytical tools comprise radon transformation.

In one embodiment, the one or more analytical tools comprise HOS techniques to generate a bispectrum plot and/or a cumulant plot.

In one embodiment, the one or more analytical tools comprises radon transformation, HOS techniques, and active contour models.

In one embodiment, the at least one ultrasound image comprises an M-mode image, wherein the one or more analytical tools comprise radon transformation, HOS techniques and active contour model.

In one embodiment, the at least one ultrasound image comprises an M-mode image, wherein the one or more analytical tools comprise radon transformation and HOS techniques.

In one embodiment, the at least one ultrasound image comprises an M-mode image, wherein the one or more analytical tools comprise active contour model.

In one embodiment, the anatomical structure is a heart or blood vessel or other internal body organ of a subject.

In one embodiment, the blood vessel is the brachial artery.

In one embodiment, the at least one ultrasound patch is connected to the server through a wireless connection.

In one embodiment, there is provided a computed implemented method for automatically assessing an anatomical structure of a subject, comprising: obtaining at least one ultrasound image from an ultrasound patch; transmitting the at least one ultrasound image into a server comprising a cloud system; processing the at least one ultrasound image in the cloud system using one or more analytical tools to generate at least one processed ultrasound image; inputting the at least one processed ultrasound image into a deep learning CNN to obtain an automatic classification result selected from two or more classes indicating the functional state of the anatomical structure; and displaying the classification result to a user.

In one embodiment, the at least one processed ultrasound image is classified into two classes of either “normal” or “abnormal”.

In one embodiment, the classification result is indicative of the subject's likelihood of having a condition or disease.

In one embodiment, the classification result identifies at least one of damaged tissue, blockages to blood flow, narrowing of vessels, tumors, congenital vascular malformations, reduced blood flow, absent blood flow or increased blood flow.

In one embodiment, the condition or disease is at least one of cardiovascular disease, cancer, infection or soft tissue damage.

In one embodiment, the least one ultrasound image is transmitted to the server through a wireless connection.

In one embodiment, there is provided a method of assisting in identifying an ailment or determining a prognosis of a subject with an ailment, the method comprising steps of: obtaining at least one ultrasound image of an anatomical structure in the subject from at least one ultrasound patch attached to the subject; transmitting the at least one ultrasound image into a server; processing the at least one ultrasound image using one or more analytical tools to generate at least one processed ultrasound image; inputting the at least one processed ultrasound image into a deep learning CNN to obtain an automatic classification result selected from two or more classes indicating the functional state of the anatomical structure, and displaying the classification result to a user, wherein the classification result is indicative of the subject's risk of having an ailment or the prognosis of an ailment.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the disclosure is not limited to specific methods and instrumentalities disclosed herein. Moreover, those in the art will understand that the drawings are not to scale. Wherever possible, like elements have been indicated by identical numbers.

FIG. 1 is a schematic diagram of a computed implemented system 100 for automated monitoring of an anatomical structure of a subject using an ultrasound patch 110, in accordance with the disclosed embodiments.

FIG. 2 is a block diagram of the flexible printed circuit board 200 of the ultrasound patch 110, in accordance with the disclosed embodiments.

FIG. 3 is a schematic diagram showing the steps for classifying the input ultrasound images using the CNN network.

FIG. 4 is a flow diagram showing an exemplified embodiment of the processing of an ultrasound image obtained from a brachial artery of a subject for classification of the functional state.

FIG. 5 shows cumulant plots and bispectrum plots processed from time-series data of ultrasound signals of brachial artery acquired at Normal, Occlusion and Release functional states during brachial artery occlusion tests in 5 subjects.

FIG. 6 shows segmented images processed using the active contour method of the brachial artery at Normal, Occlusion and Release functional states during brachial artery occlusion tests in 5 subjects.

FIG. 7 shows attention maps of the brachial artery at Normal, Occlusion and Release functional states during brachial artery occlusion tests in 5 subjects.

### NUMERICAL REFERENCE FEATURES

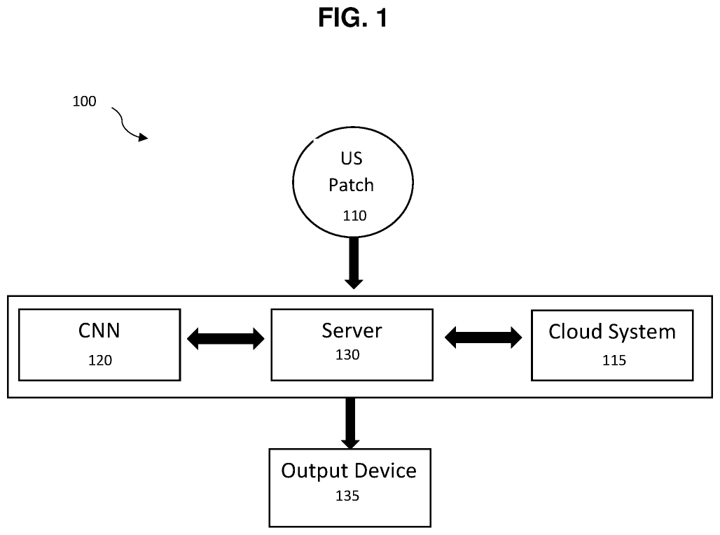

The following list of index numbers and associated features is intended for ease of reference to FIG. 1 through FIG. 7 and illustrative embodiments of the disclosure:
100—system for automated monitoring of an anatomical structure of a subject using an ultrasound patch
110—ultrasound patch
115—cloud system
120—CNN model
130—server
135—output device
200—circuit board
205—sensor
210—pulser/receiver
215—microprocessor
220—power source
225—transmitter/receiver (Tx, Rx)
230—antenna/communication system

### Definitions

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

As applicable, the terms “about” or “generally”, as used herein in the specification and appended claims, and unless otherwise indicated, means a margin of +/−10%. Also, as applicable, the term “substantially” as used herein in the specification and appended claims, unless otherwise indicated, means a margin of +/−20%. It is to be appreciated that not all uses of the above terms are quantifiable such that the referenced ranges can be applied.

The term “anatomical structure” or “structure” refers to any part of the human body, typically a component of an anatomical system, such as organs, tissues, and cells. In this regard, tissue may refer to any body tissue including but are not limited to muscle tissue, connective tissue, epithelial tissue and nervous tissue in the body of a subject. For example, the system described herein can monitor an anatomical structure such as soft tissue (e.g. for inserting a catheter/needle), pulmonary tissue (e.g. artery/vein), heart (e.g. for hemoperitoneum and pericardial tamponade), abdomen (including the pancreas, aorta, inferior vena cava, liver, gall bladder, bile ducts, kidneys, and spleen), female pelvic organs (e.g. uterus, ovaries, and Fallopian tubes), the bladder, adnexa, Pouch of Douglas, head and neck (including thyroid and parathyroid glands, lymph nodes, and salivary glands), and musculoskeletal system (including tendons, muscles, nerves, ligaments, soft tissue masses, and bone surfaces).

The term “condition” or “disease” can be used interchangeably with “ailment” and generally refer to an illness, disease or other physical or mental disorder. Ailments that can be identified by ultrasound include, for example, arterial and venous disease, peripheral vascular disease, cardiac stenosis or insufficiency, gastroenterology and colorectal abnormalities, abnormalities of the pancreas, aorta, inferior vena cava, liver, gall bladder, bile ducts, kidneys, and spleen, appendicitis, abnormalities of the thyroid and parathyroid glands, lymph nodes, and salivary glands. Abnormalities can include damaged tissue/trauma, blockages to blood flow (such as clots), narrowing of vessels, tumors and congenital vascular malformations, reduced or absent blood flow to various organs, such as the testes or ovary increased blood flow, which can be a sign of infection.

The term “subject”, “patient” and “individual” are used interchangeably herein, and refer to an animal, for example, a human or no-human animal that the ultrasound patch can be attached to for receiving ultrasound images. For measuring or monitoring of conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like. The patients, individuals or subjects of the invention system and method are, in addition to humans, veterinary subjects in the context of this disclosure herein. Such subjects include livestock and pets as well as sports animals such as horses, greyhounds, and the like.

The term “deep learning” refers to a refinement of artificial neural network (“ANN”), consisting of more than one hidden layer that permits higher levels of abstraction and improved predictions from data. A “deep learning model” refers to classification models that can include deep learning neural network models.

The term “convolutional neural network” (“CNN”) is as conventionally used in the technical field and generally refers to powerful tools for computer vision tasks, whereby deep learning CNNs can be formulated to automatically learn mid-level and high-level abstractions obtained from raw data such as images. A convolution layer can contain one or more convolution kernels, which each have an input matrix, which can be the same, but which have different coefficients corresponding to different filters. Each convolution kernel in a layer produces a different output map such that the output neurons are different for each kernel. The convolutional networks can also include local or global “pooling” layers which combine the neuron group outputs of one or more output maps. The combination of the outputs can consist, for example, in taking the maximum or average value of the outputs of the group of neurons, for the corresponding output, on the output map of the “pooling” layer. The “pooling” layers make it possible to reduce the size of the output maps from one layer to the other in the network, while improving the performance levels thereof by making it more tolerant to small deformations or translations in the input data.

The term “computer learning” refers to an application of artificial intelligence (AI) that provides systems the ability to automatically learn and improve from experience without being explicitly programmed.

The term “module” refers to a self-contained unit, such as an assembly of electronic components and associated wiring or a segment of computer software, which itself performs a defined task and can be linked with other such units to form a larger system.

The term “M-mode” refers to the time motion display of the ultrasound readout along a single chosen path of the ultrasound beam. The ultrasound readout is typically the spatial information about the depth of organ boundaries that produce sound wave reflections as they move relative to the path of the ultrasound beam that emanates from the ultrasound source. The ultrasound readout can also comprise the above-mentioned one-dimensional spatial depth information combined with color-coded Doppler-derived velocity information, i.e. color M-mode.

The terms “two-dimensional” or “2D” or “three-dimensional” or “3D” in the context of ultrasound refer to the spatial dimensions of the ultrasound image. The 2D or 3D ultrasound image may be one that is constructed from the fundamental or transmitted ultrasound frequency. Alternatively, better quality images may be constructed from the harmonic frequencies that are generated from the non-linear propagation of ultrasound through the body tissues.

The terms “Doppler” and “Doppler ultrasound” refer to the use of the Doppler effect to calculate and visualize the velocity of blood flow or (in the case of tissue Doppler imaging) tissue in the structure of interest, and encompass the various modes of Doppler image acquisition and readouts. Doppler ultrasound is based on the detection of changes in frequency of ultrasound waves reflected off blood cells or tissues that are moving relative to and in the direction of the ultrasound source. Spectral Doppler ultrasound comprises continuous-wave and pulse-wave Doppler, which calculate the maximum blood velocity and specific blood velocity at the sampled depth, respectively, along the line of the ultrasound beam that are then displayed graphically with the obtained velocity in the y-axis against time in the x-axis. In color Doppler, the calculated blood velocities in an area or volume of interest are converted by a computer into an array of colors that is overlaid onto a standard 2D or 3D image of the structure of interest for color visualization of the speed and direction of blood flow within the structure. In power Doppler, which is more sensitive than color Doppler, only the speed but not direction of the blood flow is depicted. Using filters to enhance the reflected ultrasound signal from tissue and attenuate the signal from blood, tissue Doppler imaging allows the velocities from tissues in the structure of interest to be calculated. Pulse-wave tissue Doppler imaging-derived tissue velocities at a single small sampled region can be acquired at high temporal resolution and then displayed graphically with the tissue velocity on the y-axis and time on the x-axis. Alternatively, tissue velocities within a larger area or volume of interest in the structure can be acquired at lower time resolution, and the tissue velocities are then encoded within and displayed using a color-coded 2D area or 3D volume of interest in the image of the structure of interest.

The term “time-series data” refers to data acquired continuously over a predetermined time period, e.g. at least 15 seconds up to 24 hours, that is longer than the typical duration of a conventional “cine” scan. The predetermined time period may be modified dependent on the structure to monitor, the subject, and/or the circumstance of the assessment and monitoring. The “time-series data” can itself be divided into segments of smaller “time-series data” of shorter durations. “Time-series data” are typically displayed in a video format and can comprise images depicting 2D spatial information with or without an overlay of Doppler-derived velocity information acquired over time, or images depicting 3D spatial information with or without an overlay of Doppler-derived velocity information acquired over time. Where a still image depicts structural and/or velocity information over time, such as an M-mode ultrasound or spectral Doppler ultrasound readout, the image data can constitute “time-series data” over said time period. In particular, a M-mode image itself plots one-dimensional distance over time on the y-axis and x-axis, respectively, and thus represents a “time-series data” image.

The term “processor” includes any suitable hardware and/or software system, mechanism or component that processes data, signals, or other information. The processor may include a general-purpose central processing unit, a multi-processing unit, a dedicated circuit that implements a specific function, or other system. The process need not be limited to geographic locations or have time limits. For example, the processor can perform functions in “real time”, “offline”, “batch mode”, and the like. Some of the processing can be performed at different times and places by another (or the same) processing system. Examples of processing systems can include servers, clients, end-user devices, routers, switches, network storage, and the like. The computer can be any processor that communicates with memory. The memory is any suitable processor readable storage medium, such as random-access memory (RAM), read only memory (ROM), magnetic or optical disk, or other tangible medium, suitable for storing instructions to be executed by the processor.

The term “features” refers to the hidden signatures present in images.

The term “ReLu” refers to Rectified Linear Unit that is an activation function used avoid gradient exploding during training, whereby Gradient exploding refers to a model that is not trained or converging.

The term “radon transform” or “radon transformation” refers to the integral transform which takes a function f defined on the plane to a function Rf defined on the (two-dimensional) space of lines in the plane, whose value at a particular line is equal to the line integral of the function over that line. As used herein, radon transformation converts an image into one-dimensional time-series and captures directional features of an image using line integrals.

The term “active contour model” or “snake” refers to a framework in computer vision for delineating an object outline from a possibly noisy 2D image. The model is popular in computer vision, and snakes are widely used in applications like object tracking, shape recognition, segmentation, edge detection and stereo matching.

The term “radiomics” refers to a method that extracts a large number of features from radiographic medical images using data-characterization algorithms. The features, termed radiomic features, can uncover disease characteristics that would otherwise by undetected by visual inspection. A goal is to identify a “radiomic signature” which could include several features indicative of an ailment.

The term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION OF ILLUSTRATIVE EMBODIMENTS

The particular configurations discussed in the following description are non-limiting examples that can be varied and are cited merely to illustrate at least one embodiment and are not intended to limit the scope thereof.

The use of ultrasound to study anatomical structures in a subject's body such as the heart, is a common tool for diagnosing abnormal structures and/or function. Ultrasound can also be used to monitor and measure the health of an anatomical structure at rest, in response to stressors, or during surgery or other procedures. In evaluating the health of subject, clinicians and other medical personnel often require that measurements of anatomical structures be obtained and typically conduct medical tests to evaluate the function and/or response to stressors of the anatomical structure. Such measurements can be indicative of different types of medical conditions/diseases or indicative of the likelihood of such medical conditions/diseases developing.

Ultrasound can also be important when treating subjects. Clinicians and other medical personnel often require that measurements of anatomical structures be obtained during the treatment process, which can be a surgical or other interventional procedure or administration of a drug or other form of therapy. Doing so can be important to evaluate the function and/or response to the treatment. Such measurements can often be indicative of efficacy, ineffective treatment or the likelihood of a future response to such treatment, which may ultimately determine the prognosis.

As will be appreciated, generating such measurements in a manual process requires trained ultrasound sonographers to physically apply a probe to a subject. The subject must be monitored and measurements recorded and compiled into a medical report document. Manual processes are time consuming, resource intensive, subject to human error, and may result in incomplete sets of measurements due. In addition, the requirement for manual application and adjustment of the ultrasound probe limits the duration of ultrasound signal acquisition.

Thus, from a healthcare provider viewpoint, an ultrasound sensor patch removes the necessity for having a human sonographer to manually apply and adjust the probe, which facilitates in extending the duration of ultrasound signal acquisition of anatomical structure in a subject. The implementation of a wireless ultrasound patch design will also enable the ultrasound signal acquisition to be carried out remotely. Coupled with low-energy design of the ultrasound patch sensor, which obviates the need for regular recharging, continuous extended remote ultrasound monitoring in the ambulatory setting will become possible.

Thus, automated computer implemented systems that can themselves generate a complete set of measurements of anatomical structures from ultrasound images could be of great benefit in assisting with decision support services for medical professionals. Such an automated computer implemented system can accelerate the process of generating an ultrasound medical assessment and expedite workflow. From a healthcare provider viewpoint, an automated computer implemented system of this sort may remove the necessity for having a human sonographer manually measure and record measurements of anatomical structures. An automated system can improve the efficiency of the workflow leading to better diagnosis (i.e. more reliable and accurate), prognostication and treatment monitoring of the subject.

Further, automated computer implemented systems that can process and analyze ultrasound signal data efficiently can be advantageous in ultrasound signal acquisition over extended durations. Ultrasound systems that take multiple images over time will generate large volumes of data that must be processed and analyzed.

Continuous signal monitoring potentially generates voluminous data that require commensurately more time to process and analyze, which may not be feasible with manual or conventional methods of processing and/or analysis. Deep learning CNN can increase the efficiency and time cost of processing and analyzing these large volume data sets.

It will be appreciated that the present invention may be suitably adapted for use in monitoring the health of various anatomical structures conventionally monitored through ultrasound imaging, including the heart, blood vessels, lungs, joints, muscles, body tissues, and tumors of a subject. The subject can be monitored using the system disclosed herein at rest, during and after application of physiological stress conditions, including ischemia-reperfusion, exercise, heat/cold application, as well as during surgery or other procedures. The subject can also be monitored using the system disclosed herein for extended periods both in the hospital or remotely in the ambulatory setting.

Disclosed herein are a system and method that provides a user, such as a clinician or medical professional, with assistance in determining the health of an anatomical structure in a subject and subsequently the likelihood of said subject having a condition or disease. The system and method can apply one or more data analytical techniques to an obtained ultrasound image(s) that is then fed into a trained deep learning CNN to automatically classify the functional state of the anatomical structure selected from two or more classes, such as “normal” or “abnormal”. This classification can contribute towards distinguishing various functional states of the anatomical structure that can assist in distinguishing healthy and non-healthy (pathological) subjects from one another.

For example, the monitoring of a blood vessel with the system disclosed herein during an artery occlusion test on a subject can automatically classify the various functional states based on the time-series data recorded over a specified time period at various epochs comprising (1) at Normal resting state; (2) Occlusion (i.e. during application of external compressive pressure) and (3) after Release. This demonstrates the ability of the system to discriminate between normal blood flow and abnormal blood flow through said blood artery or discriminate different functional states of the blood vessels at the different measurement epochs. At the same time, the monitoring of a blood vessel with the system during an artery occlusion test on a subject can automatically classify blood vessel function as normal or abnormal based on the response of blood flow to occlusion-release, which can be used to simulate ischemia-reperfusion.

These automated classifications can be modified and/or subdivided in to two or more classes using clinical guidelines on the obtained measurements depending on the anatomical structure and functional states to be monitored. For example, the system disclosed herein can be applied to imaging of the heart, where ultrasound signal acquisition of the left ventricular wall motion can be performed at rest, during and after exercise, and automatic classifications made regarding the heart function based on the processing and analysis of the ultrasound signal at rest as well as the time-series data recorded at rest, during and after exercise.

As will be appreciated, the classification result can indicate the likelihood of a subject having a condition or disease, whereby the classification result can be a normal class (healthy) or abnormal class (pathological). The classification result can also be further subdivided to reflect specific severity or states of said condition or disease or ailment.

The disclosure herein provides a highly discriminative system and method for distinguishing ultrasound images into one or more classes indicating functional states or health (“normal” or “abnormal”) using data processing techniques and a trained deep learning CNN. The system and method can accurately and sensitively discriminate features indicative of a functional state, condition or disease. In particular, the system and method can detect a functional state and/or symptomatic pathologies of a condition or disease from an ultrasound image.

Consequently, the disclosure provides a solution for automatically monitoring the functional health of anatomical structures in a subject, with the classification results attained accurately and efficiently determining the likelihood of an individual having or at risk of having a condition or disease.

In one embodiment the system and method can automatically classify at least one ultrasound image into one or more classes that represent the functional state of the anatomical structure. In one embodiment, the classification can be divided into two classes, for example, “normal” versus “abnormal” blood vessel response to ischemia-reperfusion, which is a surrogate for endothelial function. In one embodiment, the classification can be divided into more than two classes that are either quantitative, or qualitative depending on the anatomical structure to be monitored and the clinical setting. For example, in the context of the heart as the anatomical structure, quantitative classes can include “normal” and one or more classes representing various grades of severity of functional impairment (i.e. heart contractile function), whereas qualitative classes can include “normal”, “ischemic” or “infarcted” myocardium.

In one embodiment, the system and method can automatically classify at least one ultrasound image as either normal class or abnormal class. The normal and/or abnormal classification can be further subdivided into more than one other classification depending on the anatomical structure to be monitored.

FIG. 1 shows a schematic diagram of a representative computer implemented system 100 for automated monitoring of anatomical structures in a subject body using an ultrasound patch 110. The ultrasound patch 110 includes one or more single ultrasound sensors, an electric board for ultrasound transmission/reception and communication, and a means for attachment to the subject. The sensor patch 110 transmits a burst of ultrasound and receives echo signals from the anatomical structure that is being monitored. The echo signals are used for generating at least one ultrasound image that can then be transmitted to the server 130, where a cloud system 115 is located for processing and analyzing the image data. The processed image can then be fed to a trained deep learning CNN model 120 and executed through the server 130 to automatically classify the image to obtain an automatic classification result selected from two or more classes. The classification results from the CNN are then sent to the server 130 and then displayed to one or more users via an output device 135. The classification results can be optionally validated by the user through separate monitoring devices before being communicated with the subject. The output device 135 is not limited to computer, laptop and the like.

Ultrasound Patch

In one embodiment, the system disclosed herein can include at least one ultrasound patch attached to a subject. Multiple ultrasound patches can be included and attached to the subject at one or more locations depending on the anatomical structure to be monitored. As can be appreciated the system can use 1, 2, 3, 4, 5, 6, 7, 8, 9, 10 or more patches simultaneously.

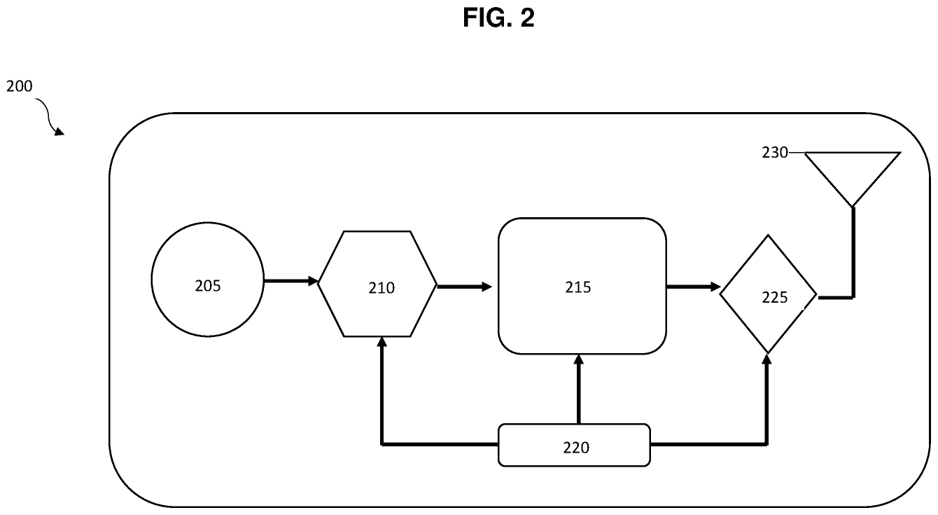

As illustrated in FIG. 2, the electrical components of the ultrasound patch can be integrated on a flexible printed circuit board 200. The components can include an ultrasound sensor 205, a pulser/receiver 210, a micro-processor 215, power source (battery) 220, a transmitter/receiver (Tx, Rx) 225 and an antenna or communication system 230.

In one embodiment, the ultrasound patch can include one or more ultrasound sensors, a microprocessor, a receiver, an electric board for ultrasound transmission and/or reception and a power source. The ultrasound patch is placed onto the surface of a subject and is “wearable”. In this regard, the patch can be shaped as a thin flexible material of plastic, elastic or fabric that can be attached to a subject with a medical tape or strap or item of apparel or accessory or combination thereof.

The one or more ultrasound sensors can transmit echo signals (image data), whereby these echo signals can be processed by the microprocessor via the pulser/receiver to generate ultrasound images.

In one embodiment, the microprocessor 215 can include an Analog to Digital (A/D) converter, digital filter and timing analyzer.

In one embodiment, the ultrasound sensor can transmit echo signals (image data) continuously and/or intermittently.

In one embodiment, the ultrasound patch can be connected to a computer server through either a wired or wireless connection for transmitting image data.

In one embodiment, the ultrasound sensor can be connected to a computer server through a wireless connection for remote monitoring with image data transmitted either continuously or intermittently. The ultrasound image data is transmitted to a receiver that transfers the image data to the server. It will be appreciated that the wireless or wired connection of the ultrasound patch disclosed herein can be through any conventional means and use of hardware components known in the technical field.

In one embodiment, the ultrasound patch can be a thin, flexible patch that operates in close contact with complex bodily surfaces of a subject. As with all applications and geometries of applied ultrasound, to perform correctly there needs to be an ‘air-free’ acoustic path for the ultrasound from the sensor surface to surface of a subject. Air cavities/bubbles would severely impede propagation of ultrasound due to their significantly lower acoustic impedance causing reflection and refraction of the propagating wave so lowering the intensity of ultrasound impinging on and propagating through the surface. A medical-grade gel can be applied to improve contact with the surface for more accurate readings.

In particular, materials applied to the surface of a subject typically employ some degree of tension in many directions to keep the material in contact with the surface. Flexible sheets of material such as paper can easily conform to singly curved shapes, e.g. cylindrical, but have difficulty in conforming to doubly curved shapes, e.g. a sphere.

As such, the ultrasound patch requires robust electrical interconnection that can withstand frequent and numerous flexing/bending. Failure of the connections could result in the patch failing to operate. Therefore, there should be an electrical interconnecting system in the ultrasound patch that can withstand repetitive bending while allowing molding and conforming to complex surface shapes. Accordingly, the ultrasound patch disclosed herein can be flexible and capable of conforming closely to surfaces of a subject's body to avoid, as much as possible, any buckling of the patch that allows air spaces to come between the patch and the subject's surface (e.g. skin).

In one embodiment, the ultrasound patch can be flexible and conforms to the surface of the subject's skin for attachment. However, it will be appreciated that in one embodiment the ultrasound patch can be modified and adapted to be attached to and conform with the surfaces of internal body cavities of a subject. Thus, the ultrasound patch can be flexible and conforms to the external (skin) or internal (body cavity) surface of the subject for attachment thereto.

In one embodiment, the external surface of the subject can be the skin.

In another embodiment, the ultrasound patch can be modified and adapted to operate as an implantable sensor.

In one embodiment, the ultrasound patch can conform closely against the external or internal surface of a subject, even though the surface is curved. In one embodiment, free-flowing gels that fill any air-spaces can be used and/or a suitable bio-compatible adhesive can be used to attach the ultrasound patch to the surface. In another embodiment, free-flowing gels that fill any air-spaces can be applied between the ultrasound patch and the surface, and the patch is secured by a suitable bio-compatible adhesive tape applied that covers the patch and surrounding surface. Alternatively, in one embodiment, the ultrasound patch can conform closely against the surface of a subject, without any gel or adhesive. The strategic use of the bio-compatible adhesive optionally can eliminate the need for application of a gel.

In one embodiment, the ultrasound patch can include one or more ultrasound sensors. In one embodiment, multiple sensor arrays can be included on a single sensor in forming any shape by the sensor pattern. In one embodiment, the ultrasound patch can form any shape including but not limited to a circle, rectangle, triangle etc. In one embodiment, the ultrasound patch can form a circular shape.

In one embodiment, the ultrasound sensor can include a piezoelectric composite transducer based on a sol-gel spray technique. This technique is a method for developing piezoelectric transducers by spraying a composite material of piezoelectric sol-gel solution and a ferroelectric powder. The sol-gel spray technique fabricates a piezoelectric layer by a sol-gel composite spraying method. The piezoelectric layer fabricated by the sol-gel composite spraying method is composed of three phases: the ferroelectric powder phase, dielectric sol-gel phase, and air phase. The air phase is generated when the alcohol and water included in the sol-gel solution vaporizes during the firing process.

In one embodiment, the ultrasound sensor can be fabricated by a sol-gel spray technique. In one embodiment, the ultrasound sensor can comprise a sol-gel composite material. In one embodiment, the ultrasound patch can include a thin and flexible piezoelectric material.

The ultrasound patch can simultaneously and continuously measure ultrasound and echo signals. In one embodiment, the ultrasound patch can generate at least one ultrasound image in one or more modes selected from the group consisting of M-mode, 2D, 3D and Doppler ultrasound.

In one embodiment, the ultrasound patch generates a 2D image.

In one embodiment, the ultrasound patch generates a 3D image.

In one embodiment, the ultrasound patch generates a M-mode image combined with a 2D image that may either be still or moving (“cine” mode) in a dual display format.

In one embodiment, the ultrasound patch generates a Doppler image.

In one embodiment, the ultrasound patch generates a Doppler image combined with a 2D image that can either be still or moving (“cine” mode) in a dual display.

In this regard, the ultrasound image can be in the form of “still” images, “cine” images of moving 2D or 3D images, or “time-series data” images. A “still” image can be a stored 2D image acquired at a finite point in time or a graphical representation of one-dimensional spatial and/or Doppler-derived velocity information plotted against acquisition time. The said graphical representation, when acquired over a predetermined period of time that is longer than that for conventional “cine” movies, can constitute “time-series data”. “Cine” movies can be formed from ultrasound echoes acquired and ultrasound images generated over a period of time, typically a few or several seconds, that is deemed sufficient to depict the phasic motion of the anatomical structure of interest. “Time-series data” can comprise image(s) for monitoring in real time structural and functional changes in the anatomical structure of interest. For example, with the application of a stressor or administration of therapy to a subject, wherein the ultrasound pulses are constantly transmitted and received to form a video over a predetermined period of time that is longer than that for conventional “cine” movies.

In one embodiment, the time-series data set can form sequential 2D moving images, or a M-mode image spectral Doppler image that inherently displays changing spatial dimension or velocity with time.

In one embodiment, the ultrasound patch in the one or more modes can generate at least one ultrasound image for the monitoring of anatomical structures.

In one embodiment, the anatomical structures for monitoring with the ultrasound patch can include blood vessels, the heart and internal body organs. The monitoring can be carried out while the subject is at rest as well as before, during and after application of physiological stress conditions, including ischemia-reperfusion, exercise or heat/cold application.

In one embodiment, the anatomical structures for monitoring with the ultrasound patch can include blood vessels, the heart and internal body organs. The monitoring can be carried out while the subject is before, during and after receiving treatment, including surgery, other interventional procedures and administration of drug therapy.

Ultrasound Image Processing

As will be appreciated, the system and method described herein can be implemented on a programmable computer using a combination of both hardware and software. Various aspects can be implemented on programmable computers, each computer including a one or more input unit, a data storage medium, a hardware processor and an output unit or communication interface. It should be appreciated that the use of terms such as servers, services, units, modules, interfaces, portals, platforms, or other systems formed from computing devices is deemed to represent one or more computing devices having at least one processor configured to execute software instructions stored on a computer readable storage medium. For example, a server can include one or more computers operating as a web server, database server, or other type of computer server in a manner to fulfil described roles, responsibilities, or functions.

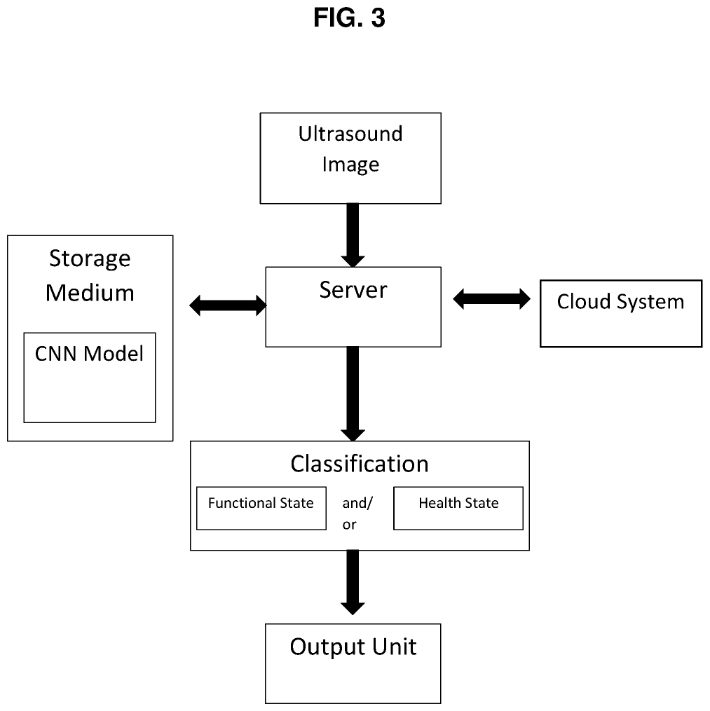

FIG. 3 is a representative flowchart of the system and operational relationship between the ultrasound patch, computational hardware and software components. The system can comprise an ultrasound patch that generates at least one ultrasound image, a server for receiving the at least one ultrasound image, a cloud system, a storage medium configured to store information defining the deep learning CNN as software instructions for execution by the server, and an output unit configured to communicate the classification result obtained from the server to one or more users.

Further, the computer system disclosed herein can include additional components. For example, the system can include one or more communication channels or interconnection mechanism such as a bus, controller, or network, that interconnects the components of the system. In various embodiments of the operating system software provides an operating environment for various software's executing in the computer system and manages different functionalities of the components of the computer system. The communication channel(s) allow communication over a communication medium to various other computing entities. The communication medium provides information such as program instructions, or other data in a communication media. The communication media can include wired or wireless methodologies implemented with an electrical, optical, radiofrequency, infrared, acoustic, microwave, bluetooth or other transmission media.

Accordingly, in one embodiment, the computer system disclosed herein can include one or more communication component for wired and/or wireless methodologies of receiving image data from the ultrasound patch. In one embodiment, the computer system disclosed herein can include one or more communication component for wireless methodologies of receiving image data from the ultrasound patch. The communication component can include a wireless receiver that transmits the ultrasound image to the server.

In one embodiment, the pulser/receiver of the ultrasound patch can transmit/receive ultrasound pulses and transforms the pulses into an ultrasound image. In one embodiment, the pulser/receiver of the ultrasound patch can transform the pulses to generate one line of M-mode and other type of images (2D, 3D or Doppler) for sending to the server.

Accordingly, in one embodiment there is provided a system for automatically monitoring anatomical structures of a subject that can comprise a ultrasound patch for generating at least one ultrasound image, a server, a cloud system, a storage medium configured to store instructions defining a deep learning CNN as software instructions for execution of the deep learning convolutional neural network to automatically classify the at least one ultrasound image, and an output unit configured to communicate the classification result to a user.

In another embodiment, there is provided a system for automatically monitoring anatomical structures of a subject that can comprise a wireless ultrasound patch for generating at least one ultrasound image, a wireless receiver, a server, a cloud system, a storage medium configured to store instructions defining a deep learning CNN as software instructions for execution of the deep learning CNN to automatically classify the at least one ultrasound image, and an output unit configured to communicate the result to a user.

The system described herein can implement a method for automatically monitoring anatomical structures of a subject. Accordingly, in another embodiment there is provided a computer-implemented method for automatically monitoring anatomical structures of a subject that can include the steps of: obtaining at least one ultrasound image from an ultrasound patch disclosed herein; inputting (transmitting) the at least one ultrasound image into a server comprising a cloud system; processing the at least one ultrasound image using one or more analytical tools to generate at least one processed ultrasound image; and inputting the at least one processed ultrasound image into a deep learning CNN to obtain an automatic classification result on the functional state of the at least one processed ultrasound image.

In another embodiment, the system described herein can implement a method of identifying an ailment or determining a prognosis of a subject with an ailment, the method can comprise the steps of: obtaining at least one ultrasound image of an anatomical structure in the subject from at least one ultrasound patch attached to the subject; transmitting the at least one ultrasound image into a server; processing the at least one ultrasound image using one or more analytical tools to generate at least one processed ultrasound image; inputting the at least one processed ultrasound image into a deep learning CNN to obtain an automatic classification result selected from two or more classes indicating the functional state of the anatomical structure, and displaying the classification result to a user, wherein the classification result is indicative of the subject having an ailment or the prognosis of an ailment. In this regard, the method can assist a user in determining the likelihood of a subject having an ailment or at risk of having an ailment based upon the classification results. The method can also assist a user in determining the susceptibility of the subject having an ailment based on the classification results. As such, the classification results can indicate to the user the increased risk or decreased risk of the subject having a certain ailment, as well as assisting in determining a prognostic outcome of said ailment.

In one embodiment, the at least one ultrasound image can be a M-mode image, 2D image, 3D image, Doppler image or a combination thereof. In an embodiment, the system is capable of processing multiple ultrasound images that represent still and/or time-series ultrasound signals.

In one embodiment, the at least one ultrasound image can be generated from a time-series data set. In this regard, the ultrasound image representing a time-series data set can be segmented and divided into segments of smaller time-series data sets of shorter time frames and durations for analysis. In one embodiment, the time-series data set can represent any time period desired for the purpose of monitoring and assessing the anatomical structure. In one embodiment, the segments can represent time intervals of 1 seconds, 5 seconds, 10 seconds, 15 seconds, 20 seconds and more, whereby it will be appreciated that any time interval for segmentation can be applied. In one embodiment, the segments can represent time intervals of 15 seconds.

In one embodiment, the at least one ultrasound image can be subjected to and processed through one or more analytical tools. The one or more analytical tools can be stored and executed in a cloud system connected to the server or in the server itself. In one embodiment, the one or more analytical tools can be stored and executed in the server without the need for a cloud system. The analytical tools can include but are not limited to radon transformation, HOS techniques, and active contour models.

In one embodiment, the at least one ultrasound image can be subjected to and processed through two or more analytical tools. In one embodiment, the at least one ultrasound image can be subjected to and processed through three or more analytical tools.

In one embodiment, the analytical tools can include radon transformation. Radon transformation can be used to reconstruct the input ultrasound image from computed tomography signals. In particular, the radon transformation can convert the input ultrasound image in to one dimensional time-series, whereby directional features of an image can be captured using line integrals.

In one embodiment, the analytical tools can include HOS techniques.

HOS techniques are powerful tools for analysis of non-linear, non-stationary, and non-Gaussian physiological signals obtained from an ultrasound patch attached to a subject. In particular, HOS is the spectral representation of higher-order statistics such as moments and cumulants of third and higher-order degrees. Analysis of ultrasound images using HOS features can help detect nonlinearity and deviations from Gaussianity. HOS techniques also result in signal noise reduction without the need to make assumptions about the linearity or otherwise of noise.

HOS techniques can refer to higher order statistics that can generate third order cumulant plots and/or bispectrum plots of the input ultrasound image. In one embodiment, the third order cumulant plots and/or bispectrum plots can be generated based on still or time-series data of ultrasound images.

Cumulant plots and bispectrum plots can be generated to yield unique features (radiomics) for disease identification (quantitative) using still images or time-series images obtained from the ultrasound patch. Various nonlinear parameters and texture features can be obtained from the HOS bispectrum and cumulant plots. These unique range of features can be used to identify various function states, conditions and diseases. For example, from M-mode images signals at 0 degree can be taken and time-series signals obtained to perform HOS analysis. However, it will be appreciated that signals at every 1 degree can be taken to improve the HOS analysis for classification performance. In this regard, features like entropies, and other nonlinear parameters can be extracted from these plots and proposed unique ranges for the output that is selected from two or more classes (i.e. abnormal and normal) of the CNN.

In one embodiment, the HOS techniques include generating a bispectrum plot of the ultrasound image.

In this regard, the bispectrum plot can include a non-parametric method that is approximated using the following equation,

B(f₁,f₂)=E[X(f₁)X(f₂)X*(f₁+f₂)]

wherein B(f₁, f₂) represents the bispectrum of signal, X(f) either represents the Fourier transform (or windowed part) of a segment or random signal, denoted by x(nT), by which n, T and E[.] symbolize the integer index, sampling interval expectation operation, respectively.

A deterministic signal is one that represents a fixed length record of the random signal, which is summable in discrete form, with the existence of its Fourier transform. For statistical accuracy, the expectation operation is to be conducted over a number of realizations. As windowing brings about spectral leakage in the Discrete Fourier Transform (DFT) process and in the event this effect can be neglected, the bispectrum of the initial random process is anticipated to be close to the approximated value, as computed by the equation above. In applying HOS techniques, subtle changes in the still or time-series data can be effectively captured.

The bispectrum plot can be described as a function involving two frequencies, in contrast to a power spectrum, which is described as a function involving one frequency. The frequency f can be normalized to be between 0 and 1, by the Nyquist frequency (a half of the sampling frequency). The bispectrum plot can be normalized to have a magnitude between 0 and 1, by the power spectra at component frequencies, indicating the extent of phase coupling between frequency components.

In one embodiment, the bispectrum plot can generate at least one bispectrum image for further processing.

In one embodiment, the HOS techniques include generating a cumulant plot of the ultrasound image.

In this regard, the cumulant plot can be used in the analysis of physiological signals derived from an ultrasound image of a subject. First and second order cumulant statistics may not be apt in easily detecting nonlinear changes in these signals. In one embodiment, the cumulant plot can be a third-order cumulant plot generated from the input ultrasound image(s).

Let {x1, x2, x3 . . . xk} denote a k dimensional multivariate signal. x1, x2, x3 . . . indicate the samples of the time-series. The first three order moments are then defined as seen below:

First Order Moment: m₁^{x}=E[x(n)] [1]

Second Order Moment: m₂^{x}(i)=E[x(n)x(n+i)] [2]

Third Order Moment: m₃^{x}(i,j)=E[x(n)x(n+i)x(n+j)] [3]

wherein EH represents the expectation operator, and i and j represent time lag parameters. The cumulants are then defined as the nonlinear combinations of moments. They are defined as seen below:

First Order Cumulant: C₁^{x}=m₁^{x} [4]

Second−Order Cumulant: C₂^{x}=m₂^{x}(i) [5]

Third Order Cumulant: C₃^{x}=m₃^{x}(i,j) [6]

In one embodiment, the cumulant plot can generate at least one cumulant image for further processing.

In one embodiment, the cumulant plot can use 3rd order cumulants to provide more information on the received ultrasound signal.

In applying these nonlinear techniques for ultrasound images, the plots obtained enable for changes to be seen more clearly and functional states of anatomical structures more readily discriminated.

In one embodiment, the HOS techniques can generate both a cumulant plot and a bispectrum plot of the ultrasound image.

In one embodiment, the cumulant plot and bispectrum plot of the input ultrasound image can be generated simultaneously with one another.

In one embodiment, the analytical tools can include radon transformation and HOS techniques that can include generating both a cumulant plot and a bispectrum plot of the ultrasound image. In one embodiment, the analytical tools can include radon transformation and HOS techniques that can include generating both at least one cumulant image and at least one bispectrum image.

In one embodiment, the analytical tools can include an active contour model to delineate the changes in the input ultrasound image(s). In one embodiment, the ultrasound image applied to the active contour model is an M-mode, 2D, 3D, Doppler image or a combination thereof.

In one embodiment, the ultrasound image applied to the active contour model is an M-mode image, wherein the active candor model can be applied for the purpose of segmenting the time-series data of the M-mode image.

In one embodiment, the active contour model can generate segmented ultrasound images for further processing.

The active contour model is an active deformable model which adapts itself to the given image, in this case an ultrasound image. The active contour model is an energy-minimizing spline which consists of many points and steered by its spline internal energy, and external constraint forces. There are generally five steps involved: (i) mean along the length of M-mode is taken, (ii) two of the highest peaks of the mean is found via the following parameters height threshold=0.50, distance=20, (iii) two lines were drawn across the image and fit using the active contour algorithm, and (iv) the active contour parameters: alpha=0.003, beta=0.012, w_line=9, w_edge=−3, gamma=0.1, max_iterations=1000, and (v) the Mid line is then calculated using the two fitted lines.

In one embodiment, the at least one ultrasound image can be subjected to and processed through radon transformation, HOS techniques, and active contour models.

In one embodiment, the ultrasound patch can generate an M-mode image, which represents a time-series data readout for the monitoring of anatomical structures, wherein the M-mode image can be subjected to analytical tools including radon transformation, HOS techniques and active contour model. The resulting segmented image from active contour model, HOS bispectrum and cumulant images can then be input into the server for further processing by a deep learning CNN.

In one embodiment, the ultrasound patch can generate an M-mode image, which represents a time-series data readout for the monitoring of anatomical structures, wherein the M-mode image can be subjected to analytical tools including radon transformation and HOS techniques. The resulting HOS bispectrum and cumulant images can then be input into the server for further processing by a deep learning CNN.

In one embodiment, the ultrasound patch can generate an M-mode image, which represents a time-series data readout for the monitoring of anatomical structures, wherein the M-mode image can be subjected to analytical tools including active contour model. The resulting segmented image from active contour model can then be input into the server for further processing by a deep learning CNN.

In one embodiment, the at least one ultrasound image processed through the analytical tools generates at least one processed ultrasound image for input into the deep learning CNN in order to subsequently produce a classification result of the at least one processed ultrasound image as an output.

In this regard, a storage medium of the system can store instructions for execution by the server of the deep learning CNN to automatically classify the at least one processed ultrasound image.

In this regard, the CNN and instructions for execution of the CNN can be in the form of a software product. The storage medium and software product stored thereon can include a number of instructions that enable the server to execute the instructions defining the CNN.

In one embodiment, the storage medium can be a non-transitory computer-readable medium having computer-readable program code stored thereon, the computer-readable program code can comprise instructions that when executed by the server, cause the server to receive and classify the at least one processed ultrasound image. The server extracts one or more features from the at least one processed ultrasound image using the deep learning CNN to classify the at least one processed ultrasound image. The server can be, for example, any type of general-purpose processor, microprocessor or microcontroller, a digital signal processing (DSP) processor, an integrated circuit, a field programmable gate array (FPGA), a reconfigurable processor, or any combination thereof. In one embodiment, the server can be a graphics processing unit (GPU) or a central processing unit (CPU).

In one embodiment there is provided a computer readable storage medium, storing non-transitory instructions for controlling a server to execute the computer-implemented method disclosed herein and CNN model that can be implemented either on the system disclosed herein or another system configured to execute the instructions defining the CNN model disclosed herein on said storage medium.

In one embodiment, the CNN and instructions for execution of the CNN can be stored in the cloud system and can include a number of instructions that enable the server to execute the instructions defining the CNN.

The CNN disclosed herein has been previously trained using a dataset of ultrasound images representing a variety of anatomical structures and related functional states and health states (healthy or pathological), and is processed using deep learning techniques to obtain a classification result as an output selected from two or more classes. The classification result can be communicated to assist one or more users to determine the functional state of the anatomical structure, or health state (abnormal or normal) as applicable to the clinical setting. The classification result can also assist one or more users to determine the likelihood or risk of a subject having a condition or disease associated with the anatomical structure being monitored.

The CNN utilizes automated feature learning to classify each input ultrasound image. By training a CNN through deep learning techniques, the CNN can be applied to the system and method disclosed herein for accurately and sensitively discriminating functional features that can indicate symptomatic features of conditions or diseases associated with the anatomical features. Accordingly, in one embodiment the CNN disclosed herein is a deep learning CNN.

In particular, the CNN can be trained with a back-propagation algorithm, and the weights adjusted to reduce errors for optimum training performance. The performance of the CNN can also be compared with other deep learning models like long short-term memory (LSTM) and auto encoders.

Applying a deep learning technique in training the CNN disclosed herein results in self-feature extraction that facilitates in capturing image features automatically rather than such features being pre-selected and pre-determined for a CNN to extract for classification. Specifically, the deep learning CNN automatically learns feature abstraction from the input ultrasound images. This automatic deep learning CNN model is desirable as it reduces the necessity of the time-consuming hand-crafting of features that would otherwise be required to pre-process the images with application-specific filters or by calculating computable features. As will be appreciated, the training of a CNN cannot be updated, and any change in the algorithm or training data requires re-optimization of the entire network.

In one embodiment, the deep learning CNN has been previously trained and developed with a dataset of ultrasound images. In another embodiment, the deep learning CNN has been previously trained and developed with a dataset of at least 200 ultrasound images for each anatomical structure. In one embodiment, the anatomical structures include blood vessels such as the brachial artery, the heart, joints, body tissue and tumor tissue.

These ultrasound images used for training include a heterogeneous cohort of patients with varied functional and health states. In particular, ultrasound images of a multitude of conditions or diseases at different stages and severity of development in the anatomical structure can be used for training purposes. Each of the ultrasound images in the dataset is pre-associated with a label indicating the functional state as two or more classes, such as “normal” (“healthy”), “abnormal” (“non-healthy”) according to the clinical setting, by qualified clinicians and medical professionals. The dataset can contain a comprehensive set of ultrasound images from a variety of subjects. The size of the dataset and wide variety of image size, resolution and quality can result in a more robust deep learning CNN model.

Following training of the deep learning CNN, the classification results and accompanying accuracy is preferably validated with a cross-validation technique on blinded data sets. In particular, the deep learning CNN can be processed with a validation set of ultrasound images that were not used for training and are a separate distinct dataset of images to the training dataset. The performance of the deep learning CNN using the validation dataset can be compared against the training dataset to determine the accuracy of the deep learning CNN disclosed herein.

As will be appreciated, the typical and conventional CNN architecture for image processing can include of a series of layers of convolution filters, interspersed with a series of data reduction or pooling layers. The convolution filters or kernels are applied to areas of the input image to detect increasingly more relevant features in the image, for example lines or circles, and then higher order features such as local and global shape and texture, both of which may represent a functional state or features symptomatic of a disease or condition of a monitored anatomical structure. These convolution filters are learned by the CNN from the training. The output of the CNN is typically one or more probabilities or class labels, which in the context of the present invention can be two classes (“normal” or “abnormal”) or more.

The CNN network disclosed herein can include three main layers: convolution, pooling, and fully-connected layers. In one embodiment, these three main layers can further comprise a series of convolution and pooling layers. Additional layers can be included such as merging layers (summation/addition/concatenate layers), flattening layer, activation function layer (rectified linear unit (RELU) layer or sigmoid layer).

In this regard, a representative internal architecture of the CNN disclosed herein can include at least three main layer types made up of a convolution layer, a pooling layer and a fully connected layer. The convolution and pooling layers can perform feature extraction, whereby the convolution layer detects features of the functional state or symptomatic of a condition or disease of anatomical structures. The fully connected layers then act as a classifier on top of these features and assigns a probability for the input image.

In obtaining a classification result of multiple classes as an output from the CNN, a Softmax activation function can be used. The Softmax activation function assigns a decimal probability to each class, whereby the probabilities of all the predicted class output adds up to 1. The likelihood of an image belonging to a class is determined by the probability value.

In one embodiment, the probability can be an output with a Softmax activation function.

In one embodiment, the probability can be an output with a sigmoid function and the value ranges from 0 to 1, whereby if the value is less than 0.5 then the probability is stated as “normal” and 0.5 or more then the probability is labelled as “abnormal”. The abnormal class can indicate a condition or disease of a subject. Each condition or disease can be at different stages and progress and/or severity of pathological development.

In one embodiment, the CNN can include one or more convolution layers, one or more pooling layer, one or more flattening layer, one or more fully connected layer, one or more merging layer, one or more activation function layer. Accordingly, in one embodiment the CNN can include ten, eleven, twelve or more layers.

In one embodiment, the ultrasound image(s) is first processed by a convolution layer with different sized kernels (filters) for interpreting the input image and can produce differently sized groups of feature maps. The feature maps in the convolution layer can be concatenated together for aggregation, analysis and feature extraction. The features extracted from the convolution layer can then be used for classification by subsequent layers.

After each convolution layer, a pooling layer can be performed to reduce the dimensionality of the image for classification. The pooling layer enables a reduction of the number of parameters and downsizes each feature map independently, reducing the height and width, but keeping the depth intact. The pooling layer slides a window over the input image and simply takes the max value in a window of a specific size and stride. A type of pooling that can be used is max-pooling that takes the max value in the pooling window and has no parameters.

In one embodiment, one or more merging layer can be included that takes in multiple inputs of similar shapes except for the concatenation axis, and returns a single output without losing any pertinent information.

In one embodiment, one or more flattening layer can be included to convert three-dimensional (3D) samples to two-dimensional (2D) samples by vectorization. For inputting the image into the fully connected layer, the output of the pooling layer can be flattened to a vector to become the input image to the fully connected layer. Flattening is simply arranging the 3D volume of the previous convolution and pooling layers into a 2D representation.

In one embodiment, the activation function layer can apply ReLu and/or Sigmoid activation function.

In one embodiment, the fully connected layers can be trained with a back-propagation algorithm, after which, half of the nodes are randomly dropped. As is readily appreciated in the art, a dropout can be applied to the CNN by a regularization technique to prevent overfitting during training, whereby at each iteration, a neuron or node is temporarily “dropped” or disabled with probability p. The hyperparameter p can be termed the dropout-rate and typically can be a number around 0.5, corresponding to 50% of the neurons or nodes being dropped out. In one embodiment, the CNN disclosed herein can comprise a dropout rate of 0.5.

In one embodiment, the output of the final fully connected step can yield a decimal probability from the output nodes. Each node can be represented by a class, whereby the probabilities of the predicted outputs add up to 1. The likelihood of an image belonging to a class is determined by the probability value. It will be appreciated that the CNN may be modified to further subdivide the classifications to other more specific classifications to distinguish functional states or condition/disease states associated with a particular anatomical structure.

The output result from the CNN disclosed herein can be communicated to one or more users through an output unit. Accordingly, in one embodiment the system can include an output unit configured to communicate the classification result of the at least one ultrasound image input into the system to the one or more users. In particular, the output unit communicates or displays on a user terminal and communicative interface the CNN classification result of the at least one ultrasound image that has been selected from two or more classes where applicable. In one embodiment, the output unit can be a graphical user interface (GUI). In one embodiment, the GUI can have the facility to load and display the input image with a ‘Diagnose’ button/function to be pressed by the one or more user that will display the output class on a ‘text panel’ of the GUI.

In one embodiment of the system, the one or more users of the system can comprise, one or more individuals, one or more patients, one or more physicians and any other concerned individual. In one embodiment, the output unit can be configured to communicate the classification results by the CNN in various formats. In one embodiment, the classification result can be communicated to the one or more users automatically via one or more communication channels.

The classification and output result of the system disclosed herein can be used to assist in determining the likelihood of subject having a condition or disease associated with the functional state of the anatomical structure, not necessarily for full automated diagnosis. In this regard, the system disclosed herein can also be used to indicate or determine the risk of a subject having a condition or disease associated with the monitored anatomical structure.

There are a number of advantages that can be derived from the system and the computer implemented method disclosed herein. For example, the dependence on clinicians for diagnostics can be reduced or eliminated, whereby individuals or technicians can use the system or method disclosed herein, to attain, independent predictions on the likelihood of a subject having a condition or disease associated with the monitored anatomical structure. Further, the system or method disclosed herein can reduce workload on clinicians or medical professionals in medical settings by expediting the efficient screening of conditions or diseases among populations at risk, so that clinicians can attend to patients already determined to be at high-risk of conditions or diseases associated with the monitored anatomical structure, thereby focusing on providing actual treatment in a time-efficient manner.

The system disclosed herein advantageously exhibits low noise and has good signal to noise ratio, whereby with more data input, the system can improve its robustness to noise and performance. Also, the system disclosed herein can eliminate or reduce possible human errors which may be caused during reading of the ultrasound signals.

In considering the foregoing, the system disclosed herein includes a wearable ultrasound patch for capturing ultrasound signals of a subject's anatomical structure, such as the heart and blood vessels, for periods of time with capability for remote wireless monitoring. Generated ultrasound image(s) derived from these signals will be transmitted to a server. The ultrasound image(s) can be processed with one or more analytical tools before being input into a CNN network that automatically classifies the ultrasound image(s) to obtain an automatic classification result selected from two or more classes dependent on the functional state of the anatomical structure as an output. The output can then be provided to clinicians or other personnel for their timely assessment and treatment of the subject.

For example, the system disclosed herein may be helpful during surgery or other procedures when close monitoring of the subject's heart condition or blood vessels functioning is critical. The system disclosed herein can be coupled with Automatic Heart Diagnosis System (AHDS) for real-time analysis that can obviate the need for routine manual interpretation, which may cut down costs significantly.

It will be appreciated that variations of the above disclosed and other features and functions, or alternatives thereof, may be desirably combined into many other different systems or applications. Also, various presently unforeseen or unanticipated alternatives, modifications, variations or improvements therein may be subsequently made by those skilled in the art which are also intended to be encompassed by the following claims.

Although embodiments of the current disclosure have been described comprehensively in considerable detail to cover the possible aspects, those skilled in the art would recognize that other versions of the disclosure are also possible.

### WORKING EXAMPLES

The following non-limiting examples are provided for illustrative purposes only to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Example 1

### Monitoring Blood Circulation for Ischemia-Reperfusion Response

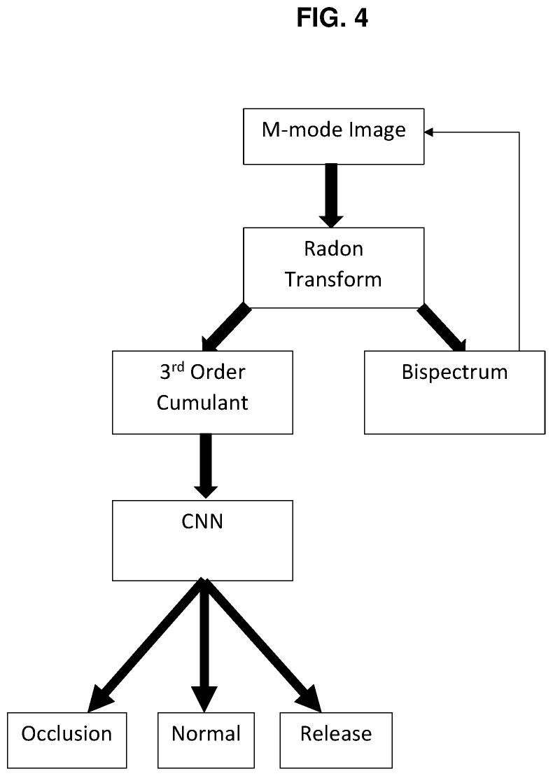

FIG. 4 shows an exemplified system disclosed herein with a subject's brachial artery being monitored during an occlusion test where abnormal functional states of a blood vessel are induced. This is to simulate the ischemia-reperfusion response to induce flow-mediated dilatation, which is a surrogate test for endothelial function. In a subject with healthy endothelial function, there is dilatation of the artery and increased blood flow during the release phase.

In this regard, to measure the diameter changes in the blood vessel in the exemplified system, ultrasound M-mode images are generated, which are inherently noisy, and the M-mode image signal can be processed into bispectrum and cumulant plots for analysis. Deep learning CNN can be applied to the processed M-mode images to generate attention maps of areas on the M-mode images with the most marked feature disparities. Through this image generation and processing, the exemplified system is able to discriminate and classify the functional state of the blood vessel as Normal, Occlusion and Release, and also the response to occlusion in individual subjects.

In this exemplified system, M-mode images of time-series readouts are obtained from the ultrasound patch and transmitted to the server and cloud system where the M-mode images are subjected to active contour method to delineate the changes in the M-mode image and then radon transformation is used to convert the M-mode image(s) into a one dimensional image. Subsequently, HOS techniques, namely HOS bispectrum and cumulant plots, are applied on the image. Then the segmented M-mode image, HOS bispectrum and cumulant images are fed to the CNN network for classification of Normal, Occlusion and Release states.

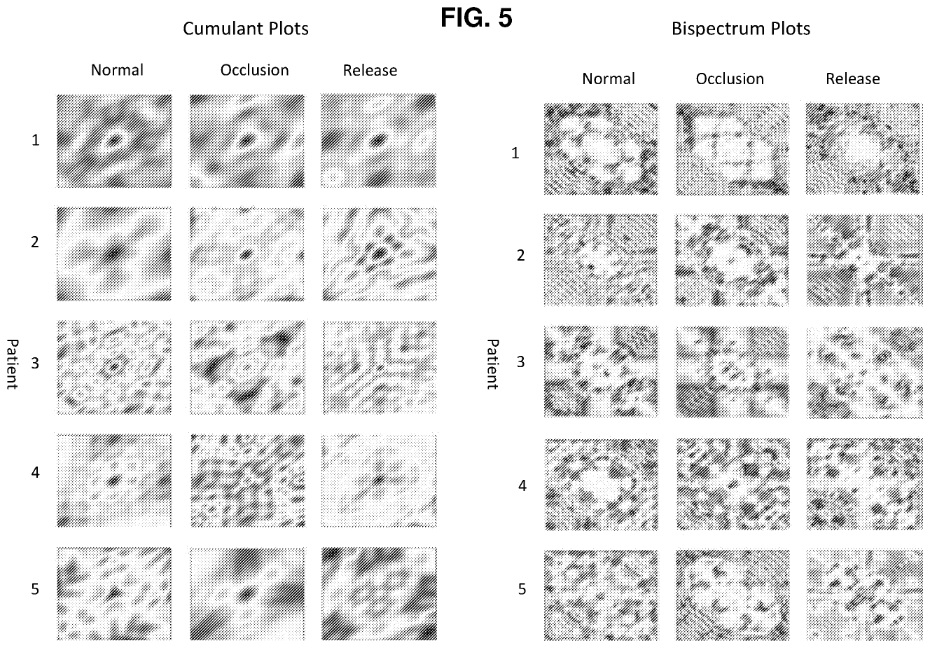

FIG. 5 shows cumulant and bispectrum plots derived from radon transformation of 15-second M-mode time-series images (without the need for conventional noise reduction, such as low-pass filter or peak detection) of 5 healthy subjects before, during and after occlusion to illustrate the differences in the Normal, Occlusion and Release phases of a brachial artery occlusion test. The cumulant and bispectrum plots in FIG. 5 demonstrate the system's ability to apply HOS techniques to discriminate between distinctive functional states of Normal (baseline, at rest), Occlusion, and Release.

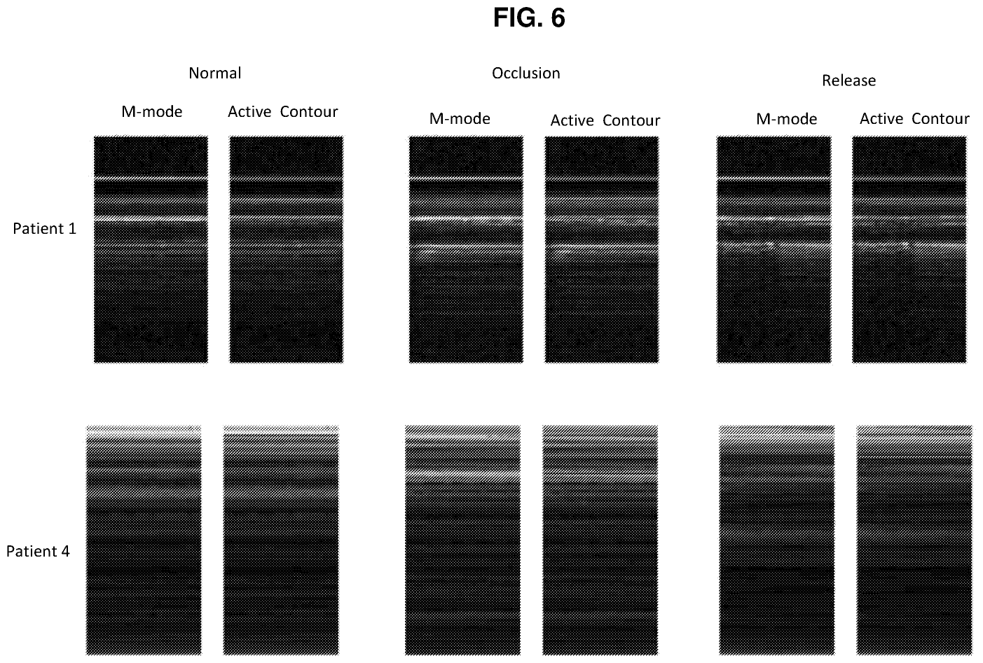

In addition, FIG. 6 shows the application of the active contour method to generate segmented images from 15-second M-mode time-series images of 5 healthy subjects before, during, and after occlusion.

Neural attention mechanism equips the CNN network with the ability to focus on a subset of its inputs (or features). Accordingly, attention and feature maps were generated from (15-second) time-series acquisitions using the trained CNN.

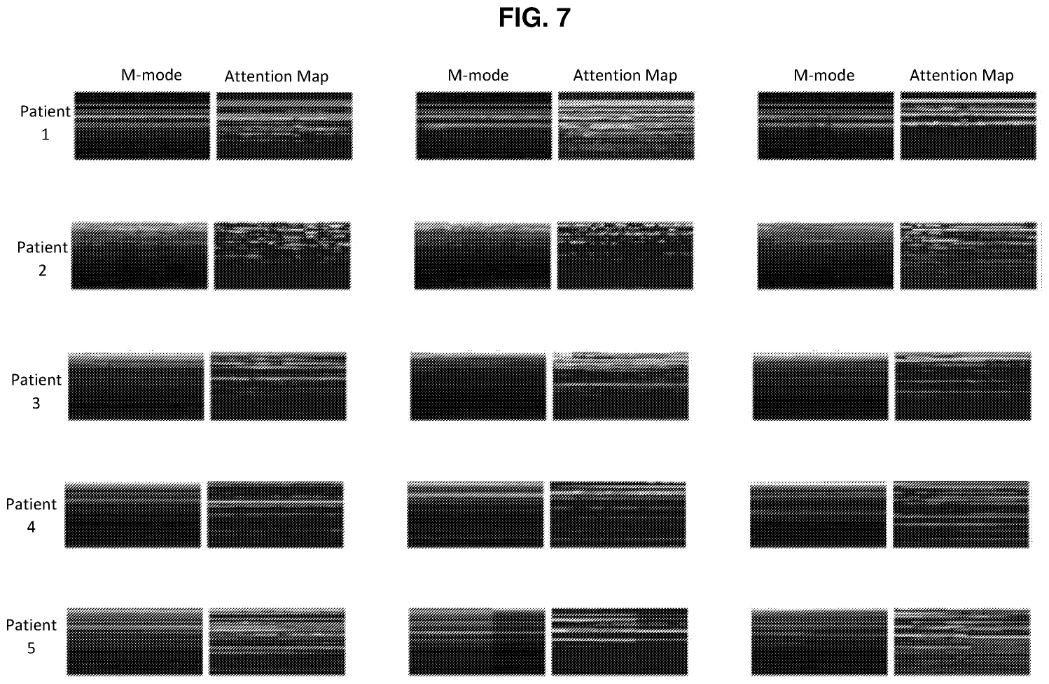

FIG. 7 shows attention maps generated from 15-second M-mode time-series images of 5 healthy subjects before, during, and after occlusion. The attention maps are derived via the last convolution layer of the network. Attention maps enable one to study the discriminative regions used by the network to identify a specific class. In this regard, attention maps are useful for debugging and can aid clinicians in understanding the decision process made by the classification CNN.

As shown in the relation to FIGS. 5, 6 and 7, M-mode time-series (15-second) images from 5 subjects were transformed into 1D image data using radon transformation. Subsequently, third order cumulant and bispectrum plots were generated for further processing in the CNN to classify the ultrasound images into 3 different classes representing functional states. It will be appreciated that in place of M-mode images, the ultrasound image may also be used as input directly without radon transformation.

Cumulant plots, bispectrum plots, and attention maps individually and/or in combination constitute unique radiomic signatures containing condensed yet entire image data (i.e. able to be transformed back to original source image and signal) that can be used for multi-parametric analyses for diagnosis and prognostication, as well as efficient data archival (for potential-omics linkage research).

It will be appreciated that the radiomic signature will be different in disease/conditions versus normal either in the baseline (resting) state or with some form of physiological alteration. Radiomic signatures are not confined to the heart and blood vessels but can also be applied to other anatomical structures such as tissues (e.g. tumor tissue) to monitor their function and motion in response to forms of temporary stressors (e.g. heat, cold, light, injection of non-specific contrast or specific ligand-modified contrast, microbubbles, ultrasound energy, radiofrequency energy, etc.).

In this regard, the system disclosed herein processes ultrasound images to obtain qualitative plots of blood vessels (i.e. brachial artery) to identify changes in functional states (i.e. blood flow occlusion) on M-mode time-series readouts generated from the ultrasound patch. Through the use of HOS techniques, the system disclosed herein is able to fully characterize and discriminate ultrasound signals and reduce noise without the assumption of linearity and Gaussian distribution either of the signal of interest or the noise. This ultimately leads to the accurate classification of processed ultrasound images as to their functional state that can indicate if the subject has a condition or disease.

### Example 2

### Monitoring Blood Circulation in the Ambulatory Setting

In this example, an elderly male smoker and diabetic patient approaches a clinician with common signs and symptoms of lower limb claudication that suggests peripheral vascular disease. Specifically, the patient experiences right calf pain after walking over 100 meters. The ankle brachial index, an established screening test for peripheral vascular disease, is more than 0.9 on the right leg, which is normal. However, it is well known that the test is insensitive and may be false negative in elderly subjects due to the relative inelasticity of arteries in the elderly.

The system and methods disclosed herein can be applied to determine the functional status of lower limb arteries. In this instance, the clinician wishes to determine dynamic changes in distal lower limb blood flow in the ambulatory setting during his routine daily activities. Accordingly, the clinician places ultrasound patches on extensor surfaces of both feet overlying the dorsalis pedis artery on both feet. The system uses ultrasound to continuously gather data that is converted to images.

The images are processed using analytical tools to show whether there is change in dorsalis pedis artery dimensions and/or blood flow at rest and with activity in the affect leg compared with the contralateral leg.

As described above, the CNN model can be trained to distinguish between healthy and abnormal lower limb circulation. Specifically, the system can apply HOS techniques to discriminate between distinctive functional states of normal versus impaired circulation. This allows the clinician to calibrate and titrate therapies. This can be done through remote wireless monitoring of heart function using ultrasound patch.

### Example 3

### Monitoring of Cardiac Function in the In- and Out-Patient Settings

In this example, a middle-aged male patient approaches a clinician with common signs and symptoms of acute decompensated heart failure. Specifically, the patient experiences shortness of breath at rest, worse on lying down, and is associated with leg edema and the blood pressure is borderline low. The patient is admitted to hospital of intravenous diuretic treatment and to initiate acute heart failure therapies. The clinician conducts a conventional echocardiogram which demonstrates poor left ventricular ejection fraction. Patient recovers from acute heart failure treatment and is subsequently discharged on chronic heart failure medications.

The system and methods of the invention are applied to determine the status of left ventricle contractile function. The clinician wishes to determine dynamic changes in left ventricular ejection fraction with the acute treatment that patient is receiving. Accordingly, the clinician places a patch on the chest of the patient close to left ventricle. The system uses ultrasound to continuously gather data that is converted to images.

The images are processed using analytical tools to show whether there is change (improvement or deterioration) or no change in the left ventricular dimensions and contractility (based on calculated ejection fraction) with treatment both in the acute phase as well as in the chronic phase after hospital discharge.

As described above, the CNN model can be trained to distinguish between healthy left ventricular function and different grades of severity of left ventricular function impairment. Specifically, the system can apply HOS techniques to discriminate between distinctive functional states of normal versus impaired left ventricular function. This allows the clinician to calibrate and titrate therapies. This can be done through remote wireless monitoring of heart function using ultrasound patch.

### Example 4

### Monitoring of Cardiac Function During Surgery

In this example, a patient with known coronary heart disease is undergoing high-risk vascular surgery of the lower limbs, which has potential to cause ischemic cardiac injury and embarrass cardiac function. The system and methods of the invention are applied to determine the status of left ventricle contractile function and wall motion continuously during surgery and in the early recovery period. Accordingly, the clinician places a patch on the chest of the patient close to left ventricle. The system uses ultrasound to continuously gather data that is converted to images.

The images show that there is no significant changes in left ventricular dimensions, left ventricular ejection fraction, left ventricular stroke volume output (stroke volume as determined by Doppler ultrasound of left ventricular outflow) and left ventricular wall motion during and early after the operation. The surgery proceeds safely, and patient recovers uneventfully.

It will be appreciated that variations of the above disclosed and other features and functions, or alternatives thereof, may be desirably combined into many other different systems or applications. Also, various presently unforeseen or unanticipated alternatives, modifications, variations or improvements therein may be subsequently made by those skilled in the art which are also intended to be encompassed by the following claims.

Although embodiments of the current disclosure have been described comprehensively in considerable detail to cover the possible aspects, those skilled in the art would recognize that other versions of the disclosure are also possible.

While the present invention has been described in terms of particular embodiments and applications, in both summarized and detailed forms, it is not intended that these descriptions in any way limit its scope to any such embodiments and applications, and it will be understood that many substitutions, changes and variations in the described embodiments, applications and details of the method and system illustrated herein and of their operation can be made by those skilled in the art without departing from the spirit of this invention.

1. A system for automatically monitoring an anatomical structure of a subject, comprising:
at least one ultrasound patch attached to said subject, wherein said patch comprises one or more ultrasound sensors, communication system, and an electric board for ultrasound transmission and/or reception, wherein the ultrasound patch generates at least one ultrasound image in one or more modes selected from the group consisting of M-mode, 2D, 3D and Doppler ultrasound;
a server for processing the at least one ultrasound image using one or more analytical tools to generate at least one processed ultrasound image, wherein the one or more analytical tools comprise radon transformation, higher-order spectra techniques, and/or active contour models;
a storage medium configured to store instructions defining a deep learning CNN,
wherein the server executes the deep learning CNN to obtain an automatic classification result selected from two or more classes indicating the functional state of the anatomical structure; and
an output to communicate the classification result to a user.

2. The system of claim 1, wherein the two or more classes comprises a normal class and abnormal class.

3. The system of claim 1, wherein the at least one ultrasound patch comprises a flexible piezoelectric material.

4. The system of claim 1, wherein the ultrasound patch is flexible and conforms to the surface of the subject.

5. The system of claim 1, wherein the ultrasound image is selected from a group of a M-mode image, doppler image, 2D image or a combination thereof.

6. (canceled)

7. (canceled)

8. The system of claim 1, wherein the one or more analytical tools comprise higher-order spectra techniques to generate a bispectrum plot and/or a cumulant plot.

9. The system of claim 1, wherein the one or more analytical tools comprises radon transformation, HOS techniques, and active contour models.

10. The system of claim 1, wherein the at least one ultrasound image comprises an M-mode image, wherein the one or more analytical tools comprises radon transformation, HOS techniques, and/or active contour models.

11. (canceled)

12. (canceled)

13. The system of claim 1, wherein the anatomical structure is a heart or blood vessel of a subject.

14. The system of claim 13, wherein the blood vessel is the brachial artery.

15. The system of claim 1, wherein the at least one ultrasound patch is connected to the server through a wireless connection.

16. A computed implemented method for automatically monitoring an anatomical structure of a subject, comprising:
obtaining at least one ultrasound image from at least one ultrasound patch;
transmitting the at least one ultrasound image into a server;
processing the at least one ultrasound image using one or more analytical tools to generate at least one processed ultrasound image;
inputting the at least one processed ultrasound image into a deep learning CNN to obtain an automatic classification result selected from two or more classes indicating the functional state of the anatomical structure; and
displaying the classification result to a user.

17. The method of claim 16, wherein the two or more classes comprises a normal class and abnormal class, and wherein the classification result is indicative of the subject's likelihood of having a condition or disease.

18. (canceled)

19. The method of claim 16, wherein the classification result identifies at least one of damaged tissue, blockages to blood flow, narrowing of vessels, tumors, congenital vascular malformations, reduced blood flow, absent blood flow or increased blood flow.

20. The method of claim 16, wherein the condition or disease is at least one of cardiovascular disease, cancer, infection or soft tissue damage.

21. The method of claim 16, wherein the at least one ultrasound image is transmitted to the server through a wireless connection.

22. A method of identifying an ailment or determining a prognosis of a subject with an ailment, the method comprising the steps of:
obtaining at least one ultrasound image of an anatomical structure in the subject from at least one ultrasound patch attached to the subject;
transmitting the at least one ultrasound image into a server;
processing the at least one ultrasound image using one or more analytical tools to generate at least one processed ultrasound image;
inputting the at least one processed ultrasound image into a deep learning CNN to obtain an automatic classification result selected from two or more classes indicating the functional state of the anatomical structure, and
displaying the classification result to a user, wherein the classification result is indicative of the subject's risk of having an ailment or the prognosis of the subject with an ailment.

23. The method of claim 22, wherein the classification result identifies at least one of damaged tissue, blockages to blood flow, narrowing of vessels, tumors, congenital vascular malformations, reduced blood flow, absent blood flow or increased blood flow.

24. The method of claim 22, wherein the ailment is at least one of cardiovascular disease, cancer, infection or soft tissue damage.

25. The method of claim 22, wherein the one or more analytical tools comprises radon transformation and/or active contour model.

26. (canceled)

27. The method of claim 22, wherein the at least one ultrasound image is transmitted to the server through a wireless connection.
